(19) United States
(12) Patent Application Publication
(10) Pub. No.: US 2007/0025973 A1
(43) Pub. Date: Feb. 1, 2007

(54) Reprogramming of adult or neonic stem cells and methods of use

(76) Inventors: Frederick J. Fitzsimmons, Wilmette, IL (US); Daniel D. Richard, Boynton Beach, FL (US)

Correspondence Address: COLEMAN SUDOL SAPONE, P.C., 714 COLORADO AVENUE, BRIDGE PORT, CT, 06605-1601, US

(21) Appl. No.: 11/495,965

(22) Filed: Jul. 28, 2006

Related U.S. Application Data
(60) Provisional application No. 60/703,834, filed on Jul. 29, 2005.
(60) Provisional application No. 60/733,324, filed on Nov. 3, 2005.

(51) Int. Cl. A61K 35/14; C12N 5/08

(52) U.S. Cl. 424/93.7; 435/372

(57) ABSTRACT

The present invention relates to methods of reprogramming adult stem cells or stem cells obtained from umbilical or placental cord blood or more preferably, the placeta or umbilical cord tissue itself or the amniotic fluid or amnion. The resulting stem cells and methods of using these stem cells represent additional aspects of the present invention. Stem cells according to the present invention, especially neonic stem cells (those obtained/reprogrammed from neonate stem cells), represent great potential in duplicating embryonic stem cell research without utilizing embryonic stems cells which have raised ethical and moral issues. The stem cells of the present invention which are reprogrammed are obtained from adults or from neonates without causing injury or death to a fetus in the case of embryonic stem cells and are non-controversial. In this sense, the present invention avoids many of the legal, ethical or moral considerations which have complicated the use of embryonic stem cells.

### RELATED APPLICATIONS

This application claims the benefit of priority of U.S. Provisional Application Ser. No. 60/703,834, filed Jul. 29, 2005, and U.S. Provisional Application Ser. No. 60/733,324, filed Nov. 3, 2005, entitled “Reprogramming of Adult or Neonic Stem Cells and Methods of Use”, both of which applications are incorporated by reference herein in their entirety.

### FIELD OF THE INVENTION

The present invention relates to methods of reprogramming adult stem cells or stem cells obtained from umbilical or placental cord blood or more preferably, the umbilical cord and placenta tissue and amniotic fluid and the amnion itself. The resulting stem cells, called neonic stem cells and methods of using these stem cells represent additional aspects of the present invention. Stem cells according to the present invention, especially neonic stem cells, represent great potential in duplicating embryonic stem cell research without utilizing embryonic stems cells which have raised ethical and moral issues. The stems cells of the present invention which are reprogrammed are obtained from adults or from neonates without causing injury or death to a fetus in the case of embryonic stem cells. In this sense, the present invention avoids many of the legal, ethical or moral considerations which have complicated the use of embryonic stem cells.

### BACKGROUND OF THE INVENTION

The use of stem cells to provide therapeutic advances against numerous disease states and conditions represents an ongoing therapeutic potential. For example, studies are ongoing to use stem cells in hematopoietic reconstitution, to treat cancer, to treat neurodegenerative diseases, to provide organs for transplants, to perform bone marrow transplants, to provide for neurotransplantation, cardiovascular transplantation, for gene therapy, etc. It is believed that the use of stem cells represents a promising approach given the ability of these cells to differentiate into various and diverse specific types of cells such as bone marrow, neuronal, cardiovascular, etc. To date, much of this work has centered on the use of embryonic stem cells to provide treatments for a variety of diseases. Adult stem cells as well as umbilical cord blood stem cells are also being investigated for these purposes.

Currently established human ES cell lines are derived from the inner cell mass of a human blastocyst. The blastocyst is the first stage of embryo differentiation. Typically, day-5 blastocysts are used to derive ES cell cultures. A normal day-5 human embryo in vitro consists of between 200 to 250 cells. A majority of these cells contribute to the trophectoderm. In order to derive ES cell cultures, the trophectoderm is removed, either by microsurgery or immunosurgery (antibodies used to free the inner cell mass). At this stage of development, the inner cell mass is composed of between 30 to 34 cells. (Bongso, A Handbook on Blastocyst Culture, Singpore: 1999).

By way of background, after a human oocyte is fertilized in vitro by a sperm cell, the following events occur according to a fairly predictable time line. Day 1 is approximately 18-24 hours following in vitro fertilization or intracytoplasmic sperm injection. By Day 2, approximately 24-25 hours post fertilization, the zygote undergoes the first cleavage to produce a 2-cell embryo. By Day 3, the embryo reaches the 8-cell stage known as the morula, an early stage of embryo development characterized by equal and pluripotent blastomeres. During the morula stage, the genome of the embryo begins to control its own development. Any maternal influences from the presence of mRNA and proteins in the oocyte cytoplasm are significantly reduced. By Day 4, the cells of the embryo adhere tightly to each other through a process called compaction. By Day 5, the cavity of the blastocyst is complete and the inner cell mass begins to separate from the outer layer or trophectoderm that surrounds the blastocyst. This is the first observable sign of cell differentiation in the embryo.

An advantage of the use of blastomeres, or cells taken from the morula stage embryo, in the present invention, is that the blastomeres differ from the cells from the inner cell mass (ICM) of the blastocyst, both in size of the adjacent cytoplasm and gene pattern expression. Upon removal of the zona pellucida from the morula, all cells are pluripotent, meaning they retain the ability to produce a variety of differentiated cells. Morula derived ES cells have potential to be more pluripotent than ES cells established from the ICM of a blastocyst. The transcription factor is considered a marker for pluripotency of stem cells and is first detected in the nuclei of cell morula, increasing in early blastocysts, and declines in late blastocysts, in which most protein is confined to the inner cell mass (ICM) region. (Liu, “Effect of Ploidy and Parentl Genome Composition on Expression of Oct-4 Protein in Mouse Embryos” Gene Expr. Patterns. 2004 July: 4(4): 433-41). Isolated prior to the onset of embryonic differentiation, morula derived ES cells tend to have less spontaneous differentiation, because they were isolated prior to first differentiation, whereas ES cells established from the ICM of blastocysts have already proceeded with differentiation. With the exception of humans, morula derived ES cells have been established in various other species, such as mouse, mink, and bovine. (Eistetter, “Pluripotent Embryonal Stem Cells can be Established from Disaggregated Mouse Morulae” Devel. Growth and Diff. 31, 275-282; Sukoyan, M. A.; Vatolin, S. Y.; Golubitsa, A. N.; Zhelezova, A. I.; Semenova, L. A.; Serov, O. L.; Embryonic Stem Cells Derived from Morulae, Inner Cell Mass, and Blastocysts of Mink: Comparisons of their Pluripotencies, Mol. Reprod. Dev. 1993 October 36(2): 148-58; Stice, S. L.; Strelchenko, N. S.; Keefer, C. L.; Matthews, L.; Pluripotent Bovine Embryonic Stem Cell Lines Direct Embryonic Developments Following Nuclear Transfer, Biol Reprod. 1996 January; 54(1): 100-110; Strelchenko, N.; Stice, S.; WO 95/16770, Ungulate Preblastocyst Derived Embryonic Stem Cells and thereof to Produce Cloned Transgenic and Chimeric Ungulates). While, this approach produces cells which are more pluripotent than cells derived from the blastocyst stage, making the present ES cell lines highly useful in cell therapy, moral, ethical and legal issues remain.

Work has recently been performed using stem cells obtained from bone marrow to provide neural cells which can be used in neuronal transplantation. See WO 99/56759. This patent represented the culmination of more than 130 years of work in the use of bone marrow stem cells for non-hematopoietic uses.

Several groups of investigators since 1990 have attempted to prepare more homogenous populations of stem cells from bone marrow. For example, U.S. Pat. No. 5,087,570, issued Feb. 11, 1992, discloses how to isolate homogeneous mammalian hematopoietic stem cell compositions. Concentrated hematopoietic stem cell compositions are substantially free of differentiated or dedicated hematopoietic cells. The desired cells are obtained by subtraction of other cells having particular markers. The resulting composition may be used to provide for individual or groups of hematopoietic lineages, to reconstitute stem cells of the host, and to identify an assay for a wide variety of hematopoietic growth factors.

U.S. Pat. No. 5,633,426 issued May 27, 1997, is another example of the differentiation and production of hematopoietic cells. Chimeric immunocompromised mice are given human bone marrow of at least 4 weeks from the time of implantation. The bone marrow assumed the normal population of bone marrow except for erythrocytes. These mice with human bone marrow may be used to study the effect of various agents on the proliferation and differentiation of human hematopoietic cells.

U.S. Pat. No. 5,665,557, issued Sep. 9, 1997, relates to methods of obtaining concentrated hematopoietic stem cells by separating out an enriched fraction of cells expressing the marker CDw 109. Methods of obtaining compositions enriched in hematopoietic megakaryocyte progenitor cells are also provided. Compositions enriched for stem cells and populations of cells obtained therefrom are also provided by the invention. Methods of use of the cells are also included.

U.S. Pat. No. 5,453,505 issued on Jun. 5, 1995, is yet another method of differentiation. Primordial tissue is introduced into immunodeficient hosts, where the primordial tissue develops and differentiates. The chimeric host allows for investigation of the processes and development of the xenogeneic tissue, testing for the effects of various agents on the growth and differentiation of the tissue, as well as identification of agents involved with the growth and differentiation.

U.S. Pat. No. 5,753,505 issued May 19, 1998, provides an isolated cellular composition comprising greater than about 90% mammalian, non-tumor-derived, neuronal progenitor cells which express a neuron-specific marker and which can give rise to progeny which can differentiate into neuronal cells. Also provided are methods of treating neuronal disorders utilizing this cellular composition.

U.S. Pat. No. 5,759,793 issued Aug. 6, 1996, provides a method for both the positive and negative selection of at least one mammalian cell population from a mixture of cell populations utilizing a magnetically stabilized fluidized bed. One application of this method is the separation and purification of hematopoietic cells. Target cell populations include human stem cells.

U.S. Pat. No. 5,789,148 issued Aug. 4, 1998, discloses a kit, composition and method for cell separation. The kit includes a centrifugable container and an organosilanized silica particle-based cell separation suspension suitable for density gradient separation, containing a polylactam and sterilized by treatment with ionizing radiation. The composition includes a silanized silica particle-based suspension for cell separation which contains at least 0.05% of a polylactam. and preferably treated by ionizing radiation. Also disclosed is a method of isolating rare blood cells from a blood cell mixture.

Within the past several years, mesenchymal stem cells (MSCs) have been explored as vehicles for both cell therapy and gene therapy. The cells are relatively easy to isolate from the small aspirates of bone marrow that can be obtained under local anesthesia: they are also relatively easy to expand in culture and to transfect with exogenous genes. Prockop, D. J. Science 26: 71-74 (1997). Therefore, MSCs appear to have several advantages over hematopoietic stem cells (HMCs) for use in gene therapy. The isolation of adequate numbers of HSCs requires large volumes of marrow (1 liter or more), and the cells are difficult to expand in culture. (Prockop, io D. J. (ibid.)).

There are several sources for bone marrow tissue, including the patient's own bone marrow, that of blood relatives or others with MHC matches and bone marrow banks. There are several patents that encompass this source. U.S. Pat. No. 5,476,997 issued May 17, 1994, discloses a method of producing human bone marrow equivalent. A human hematopoietic system is provided in an immunocompromised mammalian host, where the hematopoietic system is functional for extended periods of time. In this method, human fetal liver tissue and human fetal thymus are introduced into a young immunocompromised mouse at a site supplied with a vascular system, whereby the fetal tissue results in formation of functional human bone marrow tissue.

Human fetal tissue also represents a source of implantable neurons, but its use is quite controversial. U.S. Pat. No. 5,690,927 issued Nov. 25, 1997, also utilizes human fetal tissue. Human fetal neuro-derived cell lines are implanted into host tissues. The methods allow for treatment of a variety of neurological disorders and other diseases.

U.S. Pat. No. 5,753,491, issued May 19, 1998, discloses methods for treating a host by implanting genetically unrelated cells in the host. More particularly, the present invention provides human fetal neuro-derived cell lines, and methods of treating a host by implantation of these immortalized human fetal neuro-derived cells into the host. One source is the mouse, which is included in the U.S. Pat. No. 5,580,777 issued Dec. 3, 1996. This patent features a method for the in vitro production of lines of immortalized neural precursor cells, including cell lines having neuronal and/or glial cell characteristics, comprises the step of infecting neuroepithelium or neural crest cells with a retroviral vector carrying a member of the myc family of oncogenes.

U.S. Pat. No. 5,753,506 issued May 19, 1998, reveals an in vitro procedure by which a homogeneous population of multipotential precursor cells from mammalian embryonic neuroepithelium (CNS stem cells) is expanded up to 10 fold in culture while maintaining their multipotential capacity to differentiate into neurons, oligodendrocytes, and astrocytes. Chemical conditions are presented for expanding a large number of neurons from the stem cells. In addition, four factors—PDGF, CNTF, LIF, and T3—have been identified which, individually, generate significantly higher proportions of neurons, astrocytes, or oligodendrocytes. These procedures are intended to permit a large-scale preparation of the mammalian CNS stem cells, neurons, astrocytes, and oligodendrocytes. These cells are proposed as an important tool for many cell- and gene-based therapies for neurological disorders. Another source of stem cells is that of primate embryonic stem cells. U.S. Pat. No. 5,843,780 issued Dec. 1, 1998, utilizes these stem cells. A purified preparation of stem cells is disclosed. This preparation is characterized by the following cell surface markers: SSEA-I (−); SSEA-3 (+), TRA-1-60 (+); TRA-1-81 (+); and alkaline phosphatase (+). In one embodiment, the cells of the preparation have normal karyotypes and continue to proliferate in an undifferentiated state after continuous culture for eleven months. The embryonic stem cells lines are also described as retaining the ability to form trophoblasts and to differentiate into tissues derived from all three embryonic germ layers (endoderm, mesoderm and ectoderm). A method for isolating a primate embryonic stem cell line is also disclosed in the patent.

There is substantial evidence in both animal models and human patients that stem cell transplantation is a scientifically feasible and clinically promising approach to the treatment of neurodegenerative diseases and stroke as well as for repair of traumatic injuries to brain and spinal cord. Nevertheless, alternative cell sources and novel strategies for differentiation are needed to circumvent the numerous ethical and technical constraints that now limit the widespread use of neural transplantation. In short, there is a need for further development of readily available reliable sources of neural cells for transplantation.

The use of umbilical cord blood for use in hematopoietic reconsitution has been around since the work of Ende in the early 1970's. Because umbilical cord blood is rich in hematopoietic precursors, including stem cells, it represents a good source of cells for hematopoietic reconstitution. To date, however, little work has been done on using pluripotential stem cells or related neural precursors which are found in umbilical cord blood for neuronal transplantion perhaps because of the failure to realize the viable source of neuronal precursors which can be found in umbilical cord blood.

Human cord and placental blood provides a rich source of hematopoietic and other stem cells. On the basis of this finding, umbilical cord blood stem cells have been used to reconstitute hematopoiesis in children with malignant and nonmalignant diseases after treatment with myeloablative doses of chemoradiotherapy. Sirchia and Rebulla, 1999 Haematoloica 84:738-47. Early results show that a single cord blood sample provides enough hematopoietic stem cells to provide short- and long-term engraftment, and that the incidence and severity of graft-versus-host disease has been low even in HLA-mismatched transplants. These results, together with our previous discovery that bone marrow cells contain stem cells capable of differentiating into neurons and glia, led to the present invention which uses cord blood or mononuclear cell fractions thereof to repair neuronal damage in brain and spinal cord. Sanchez-Ramos, et al. 1998. Movement Disorders 13(s2): 122 and Sanchez-Ramos, et al., (2000) Exp. Neurol.

Thus, the art shows the reality and potential for the use of stem cells in treating a host of disease states and conditions. Embryonic stem cells have been posited as being particularly appropriate for being used in many of these therapies, primarily because embryonic stem cells are viewed as being very early in development and consequently, their early stage development allows for great diversity in differentiation passed through generations. However, the use of embryonic stem cells creates moral and ethical dilemmas because the basic technology requires a supply of stem cells from embryos, a supply which often results in the death of the embryo. The present invention relates to non-controversial stem cells which are reprogrammed to function as substitutes for controversial embryonic stem cells.

### BRIEF DESCRIPTION OF THE INVENTION

The present invention relates to the discovery that non-controversial stem cells which are obtained from adults (adult stem cells) or from umbilical cords or umbilical cord/placental blood, or placental tissue, amniotic fluid or amnion are capable of being reprogrammed and used as substitute stem cells for controversial embryonic stem cells.

In this first aspect of the present invention, human adult stem cells or alternatively, stem cells obtained from otherwise discarded umbilical cords themselves or alternatively, umbilical cord or placental blood, placental tissue, amniotic fluid or amnion, which is obtained after and as a consequence of the delivery of a baby, may be reprogrammed to produce cells which may be used in autologous and/or allogeneic procedures. The resulting stem cells, which have characteristics consistent with autologous embryonic stem cells, may develop and differentiate into any type of cell useful for treatment.

In preferred aspects of the present invention, the reprogrammed stem cells which were obtained directly from the umbilical cord itself or the blood of the umbilical cord or placenta, placental tissue, amniotic fluid or amnion are referred to as “neonic” stem cells. These stem cells, due to the reprogramming, are autologous to the source cells, as a consequence of reprogramming that simply creates embryonic stem cells from those original stem cells through exposure to a cell medium which is consistent with the makeup of the cytosol from embryonic stem cells, or alternatively, by fusing genetic material from an adult stem cell (or other adult cell) with a denucleated stem cell obtained room the umbilical cord, or umbilical or placental blood or placental tissue, amniotic fluid or amnion to produce a neonic stem cell. The resulting reprogrammed stem cells are autologous to the host from which the reprogramming nuclear material came from and have similar characteristics with embryonic stem cells. An exciting feature of these neonic stem cells is the fact that these stem cells have characteristics consistent with embryonic stem cells, can be developed or differentiated into virtually any other cell used in treatment and this result can be obtained without the moral, legal or ethical dilemmas posed by the use of traditional embryonic stem cells.

The present invention also relates to methods of reprogramming stem cells to produce early stage stem cells which are similar in characteristics to embryonic stem cells. In a first method, human adult stem cells which may be obtained from any adult source or stem cells which are obtained directly from umbilical cords themselves or from umbilical cord or placental blood, placental tissue, amniotic fluid or amnion are exposed to an effective concentration of cellular medium (embryonic stem cell medium) which contains growth factors and related nutrients of the cytosol of embryonic stem cells. In this method, the isolated stem cells are exposed to embryonic stem cell medium in an amount and for a period (preferably, at least through one generation of development, more preferably at least two or three generations of development) and then are isolated as reprogrammed stem cells. In the case of adult stem cells, these cells may obtain characteristics which are unexpectedly close to the phenotypic characteristics of embryonic stem cells. In the case of neonic stem cells (i.e. those stem cells which are obtained from umbilical cords or umbilical cord or placental blood, placental tissue, amniotic fluid or amnion and reprogrammed), these are phenotypically very close to embryonic stem cells.

In a second method aspect of the present invention, pluripotent stem cells obtained from umbilical cords directly, or from umbilical cord or placental blood, placental tissue, amniotic fluid or the amnion itself are first denucleated (using a centrifugation step to remove the DNA material from the cells) and then the denucleated cells are fused with cells or genetic material taken from an individual to be treated, for example a child or an adult, preferably, but not necessarily stem cells of that individual. These fused or hybrid cells obtain characteristics which are consistent with the phenotype of the pluripotent stem cells and act as pluripotent stem cells, but are autologous to the patient (have the same genotype as the individual from whom the nuclear material was taken and fused with the stem cell). The resulting hybrid stem cell or neonic stem cell may thereafter be used as a replacement for embryonic stem cells, but with the added benefit that the neonic stem cells also are essentially genotypically identical to the individual from whom the genetic material was taken and fused with the pluripotent stem cell. This results in a non-controversial stem cell which acts similar to an embryonic stem cell, limits or avoids unfavorable immunogenic responses because the stem cells are autologous (genetically identical to the host or patient) cells). Note that this approach may be taken with pluripotent stem cells or progenitor cells in order to create stem cells or progenitor cells which are particularly useful for therapy in an individual.

The reprogrammed stem cells which are obtained using the nuclear fusion technique may also be exposed to embryonic stem cell medium as discussed above in order to further reprogram the neonic cells to a level which is even closer phenotypically to an embryonic stem cell.

The reprogrammed stem cells of the present invention may be used directly in therapeutic methods for treating a variety of disease states and conditions or alternatively, these stem cells may be further differentiated into later stage (further developed) stem or progenitor cells and those differentiated cells used in therapeutic methods.

In another aspect of the present invention, there is presented a method for obtaining pluripotent stem cells from umbilical cord or placenta blood or from the umbilical cord itself, placental tissue, amniotic fluid or amnion and then exposing the stem cells to cytosolic stem cell medium, the method comprising the steps of obtaining an umbilical cord or umbilical cord or placenta blood, placental tissue, amniotic fluid or amnion, selecting pluripotential stem cells or progenitor cells from those samples and incubating the umbilical cord, placental tissue, amniotic fluid or amnion derived stem cells or progenitor cells with a cytosolic stem cell medium to reprogram the cells to a condition which is in an early stage of development and consistent with characteristics of embryonic stem cells cells to produce a population of stems cells which may be further differentiated into numerous and varied other cells, which are capable of being transplanted or used to treat disease states. The steps of the method may also be changed such that all of the cells (for example, from the umbilical cord itself, an umbilical or placental blood and/or placental tissue, amniotic fluid or amnion sample or a mononuclear cell fraction thereof) are incubated with a differentiation agent prior to separation of the neonic (reprogrammed) stem cells.

The method of the present invention may include the step of separating the pluripotential stem cells from a population of mononuclear cells obtained from the umbilical cord, umbilical cord blood, placental blood, placental tissue, amniotic fluid or amnion using a magnetic cell separator to separate out all cells which contain a CD or other marker reflective of a cell other than a pluripotent stem cell, and then expanding the cells which do not contain a marker in an embryonic stem cell medium. In the present method, the separation and incubation (differentiation) steps, may be interchanged.

Alternatively, an enriched cell population of pluripotent stem cells may be obtained from a population of mononuclear cells obtained from umbilical tissue, umbilical cord or placental blood, placental tissue, amniotic fluid or amnion by subjecting the mononuclear population to an amount of an anti-proliferative agent (such as Ara-C [cytidine arabinoside] or methotrexate, among others) effective to eliminate all or substantially all proliferating cells and then exposing the remaining non-proliferating cells to a mitogen such as epidermal growth factor or other mitogen (including other growth factors) to provide a population of differentiated cells and quiescent cells (pluripotent stem cells) which population is grown in embryonic stem cell medium such that the quiescent cells are concentrated in the cell population to greatly outnumber the differentiated cells. These resulting reprogrammed stem cells are neonic stem cells.

These same methods may be applied to human adult stem cells, with the procedure simply involving obtaining and isolating human adult stem cells, from, for example, skin, adipose, nasal, bone marrow, peripheral blood, liver, pancreas, testicles, teeth, or other tissue, preferably, although not exclusively, using non-invasive techniques, and then growing the stem cells through at least one generation, preferably at least two or three generations in embryonic stem cell medium to provide reprogrammed adult stem cells, which have phenotypic characteristics similar to embryonic stem cells.

The neonic pluripotent stem cells obtained may then be grown in a cell medium containing a differentiation agent as generally described in the art in order to change the phenotype of the stem and/or progenitor cells to neuronal and/or glial cells, organ tissue, cardiovascular cells which may be used in transplantation procedures or to treat numerous disease states directly without further purification.

The adult stem cells from any adult source, or umbilical or placental tissue, umbilical cord or placental blood, amniotic fluid or amnion sample from which the pluripotent stem and/or progenitor cells are obtained may be a fresh adult source of blood or tissue, fresh umbilical or placental tissue, or fresh umbilical cord or placental blood, or fresh cells from amniotic fluid or amnion, or reconstituted cryopreserved adult blood or tissue, umbilical cord or placental tissue, or umbilical cord or placenta blood, amniotic fluid or amnion cells or a fresh or reconstituted cryopreserved mononuclear fraction thereof.

The reprogrammed stem cells of the present invention may be used to treat cancer, including leukemia, among numerous others, a varity of neurodegenerative diseases including, for example, a neurodegenerative disorder or a brain or spinal cord injury or neurological deficit including, for example, Parkinson's disease, Huntington's disease, multiple sclerosis (MS), Alzheimer's disease, Tay Sach's disease (beta hexosamimidase deficiency), lysosomal storage disease, brain and/or spinal cord injury occurring due to ischemia, spinal cord and brain damage/injury, ataxia and alcoholism, among others, including a number which are otherwise described herein. Using the stem cells according to the invention to differentiate and grow into autologous tissues, including skin and other organs is another aspect of the present invention. Effecting hematopoietic reconstitution, to treat HIV and AIDS and other viral diseases, represents a further aspect of the invention. The use of the present reprogrammed stem cells in gene therapy represents an additional aspect of the invention.

The present invention is also directed to a method of treating neurological damage in the brain or spinal cord which occurs as a consequence of genetic defect, physical injury, environmental insult or damage from a stroke, heart attack or cardiovascular disease (most often due to ischemia) in a patient, the method comprising administering (including transplanting), an effective number or amount of neural cells and/or other cells obtained from any adult source, or umbilical cord blood, placental blood, the umbilical cord or placenta, amniotic fluid or amnion, including reconstituted cryopreserved cells and/or tissue, to said patient, including directly into the affected tissue of the patient's brain or spinal cord. Administering cells according to the present invention to a patient and allowing the cells to migrate to the appropriate site within the central nervous system and/or other body sites is another aspect of the present invention.

The following definitions are used throughout the specification to describe the present invention.

The term “patient” is used throughout the specification to describe an animal, preferably a human and/or other mammal, to whom treatment, including prophylactic treatment, with the stem cells according to the present invention, is provided. For treatment of those infections, conditions or disease states which are specific for a specific animal such as a human patient and/or other mammal, the term patient refers to that specific animal. The term “donor” is used to describe an individual (animal, including a human or other mammal) who or which donates stems cells from umbilical cords, or umbilical cord or placental blood, placental or other birth tissue, amnion tissue, amniotic tissue and/or other birth fluids for use in a patient.

The term “umbilical cord tissue”, “umbilical cord blood” or “cord blood”, “placental tissue”, “placental blood”, “amniotic fluid/cells” and “amnion/cells” is used throughout the specification to refer to tissue, cells or blood obtained from a neonate or fetus, most preferably a neonate and preferably refers to tissue and/or blood which cells or fluid is obtained from the umbilical cord, placenta or birth tissue, amnion tissue, amniotic fluid and/or other fluids of newborns. The use of cord, placental and/or amnion tissue, amnion fluid or cord or placental blood as a source of mononuclear cells is advantageous because it can be obtained relatively easily and without trauma to the donor. In contrast, the collection of bone marrow cells from a donor is a traumatic experience. Cells from the placenta and umbilical cord tissue, placental and cord blood, amniotic fluid or amnion cells can be used for autologous transplantation or allogenic transplantation, when and if needed. Pluripotent stem cells present in placental and umbilical cord blood, cord tissue, placental tissue and amniotic fluid or amnion are isolated and used in the present invention. Placeta, umbilical cord and amnion tissue is dissected and placental or cord blood is preferably obtained by direct drainage from the cord and/or by needle aspiration or other methods from the delivered placenta at the root and at distended veins, and amniotic fluid and cells are preferably obtained by direct drainage from the amniotic sac and/or by needle aspiration and/or other collection methods from the amniotic sac.

The term “effective amount” is used throughout the specification to describe concentrations or amounts of components such as differentiation agents, mitogens, stem cells, or other agents which are effective for producing an intended result including reprogramming stem cells, differentiating stem and/or progenitor cells into other cells such as neural, neuronal and/or glial cells, or effecting gene therapy, treating a cancer, a neurodegenerative disease or other neurological disease or condition including damage to the central nervous system of a patient, such as a stroke, heart attack or accident victim or for effecting a transplantation of those cells within the patient to be treated. Compositions according to the present invention may be used to effect hematopoetic reconstitution or a transplantation of neural or other cells obtained from the reprogrammed pluripotent stem cells within the composition to produce a favorable change in the brain or spinal cord, or in the disease or condition treated, whether that change is an improvement (such as stopping or reversing the degeneration of a disease or condition, reducing a neurological deficit or improving a neurological or other response) or a complete cure of the disease or condition treated.

The term “neural cells” are cells having at least an indication of neuronal or glial phenotype, such as staining for one or more neuronal or glial markers or which will differentiate into cells exhibiting neuronal or glial markers. Neural stem cells are cells with the ability to proliferate, exhibit self-maintenance or renewal over the life-time of the organism and to generate clonally related neural progeny. Neural stem cells give rise to neurons, astrocytes and oligodendrocytes during development and can replace a number of neural cells in the adult brain. Neural stem cells are neural cells for purposes of the present invention. The terms “neural cells” and “neuronal cells” are generally used interchangeably in many aspects of the present invention.

The term “administration” or “administering” is used throughout the specification to describe the process by which reprogrammed stem cells according to the present invention are delivered to a patient for treatment purposes. Reprogrammed stem cells may be administered a number of ways including parenteral (such term referring to intravenous and intraarterial as well as other appropriate parenteral routes), intrathecal, intraventricular, intraparenchymal (including into the spinal cord, brainstem or motor cortex), intracisternal, intracranial, intrastriatal, and intranigral, among others which term allows the cells to migrate to the site where needed. Reprogrammed stem cells may be administered in the form of whole cord blood or a fraction thereof (such term including a mononuclear fraction thereof or a fraction of stem cells, including a high concentration of stem cells). The compositions according to the present invention may be used without treatment with a differentiation agent (“untreated”, i.e., without further treatment in order to promote differentiation of cells within the stem cell sample) or after treatment (“treated”) with a differentiation agent or other agent which causes certain pluripotential stem and/or progenitor cells within the cord blood sample to differentiate into cells exhibiting a favorable phenotype. Administration will often depend upon the disease or condition treated and may preferably be via a parenteral route, for example, intravenously, by administration into the cerebral spinal fluid or by direct administration into the affected tissue in the brain or other body site. For example, in the case of Alzheimer's disease, Huntington's disease and Parkinson's disease, the preferred route of administration will be a transplant directly into the striatum (caudate cutamen) or directly into the substantia nigra (Parkinson's disease). In the case of amyotrophic lateral sclerosis (Lou Gehrig's disease) and multiple sclerosis, the preferred administration is through the cerebrospinal fluid. In the case of lysosomal storage disease, the preferred route of administration is via an intravenous route or through the cerebrospinal fluid. In the case of stroke, the preferred route of administration will depend upon where the stroke is, but will often be directly into the affected tissue (which may be readily determined using MRI or other imaging techniques). In the case of heart disease, the method of administration may be by direct infusion into the affected area, or it may be by the intravenous route to allow transmigration through the circulatory system and “homing” to the affected site.

The terms “grafting” and “transplanting” and “graft” and “transplantation” are used throughout the specification synonymously to describe the process by which neural and/or neuronal or other cells according to the present invention are delivered to the site within the nervous or other body system where the cells are intended to exhibit a favorable effect, such as repairing damage to a patient's central nervous system, treating a neurodegenerative disease or treating the effects of nerve, muscle and/or other damage caused by stroke, cardiovascular disease, a heart attack or physical injury or trauma or genetic damage or environmental insult to the brain and/or spinal cord or other body site(s), caused by, for example, disease, an accident or other activity. Neural and other cells for use in the present invention may also be delivered in a remote area of the body by any mode of administration as described above, relying on cellular migration to the appropriate area in the central nervous system or other body system to effect transplantation.

The term “essentially” is used to describe a population of cells or a method which is at least 95+% effective, more preferably at least about 98% effective and even more preferably at least 99% effective. Thus, a method which “essentially” eliminates a given cell population, eliminates at least about 95+% of the targeted cell population, most preferably at least about 99% of the cell population. Neural cells according to the present invention, in certain preferred embodiments, are essentially free of hematopoietic cells (i.e., the CD34+cellular component of the mononuclear cell fragment).

The term “non-tumorigenic” refers to the fact that the cells do not give rise to a neoplasm or tumor. Stem and/or progenitor cells for use in the present invention are generally free from neoplasia and cancer.

The term “cell medium” or “cell media” is used to describe a cellular growth medium in which stem cells or other cells are grown. Embryonic stem cell medium comprises at least a minimum essential medium plus optional agents such as growth factors, glucose, non-essential amino acids, insulin, transferrin and other agents well known in the art. The embryonic stem cell medium of the present invention also contains nutrients and factors which are consistent with their existence in the cytosol of a typical embryonic stem cell. In certain embodiments at least one differentiation agent may be added to the cell media to promote differentiation of certain cells within the mononuclear fraction. Exemplary media which may be used to produce neonic cells according to the present invention include, for example, in addition to the above components, one or more of fibroblast growth factor, gamma amino butyric acid, pipecholic acid, lithium and transforming growth factor beta as described in United States patent application publication no. 20060084168, relevant portions of which are incorporated by reference herein.

One of ordinary skill will readily recognize that any number of cellular media may be used to grow mononuclear cell fractions of umbilical cord blood or to provide appropriate neural proliferation media and/or differentiation media.

The term “separation” is used throughout the specification to describe the process by which pluripotent stem and/or progenitor cells are isolated from a mononuclear cell sample or a sample which contains cells other than the desirable stem and/or progenitor cells, for example, umbilical cord blood or other fragment.

The term “mitogen” is used throughout the specification to describe an agent which is added to non-proliferating cells obtained from a mononuclear cell sample in order to produce differentiated cells and quiescent cells (pluripotent stem and/or progenitor cells). A mitogen is a transforming agent which induces mitosis in certain cells other than pluripotent stem and/or progenitor cells obtained from umbilical cord blood. Preferred mitogens for use in the present invention include epidermal growth factor (EGF), among other agents such as the less preferred pokeweed mitogen, which also may be used to induce mitosis. Mitogens are also any one or a combination of a variety of growth factors which have been shown to exert mitogenic actions on neural and mesenchymal precursors. These growth factors are: Epidermal Growth Factor (EGF) family ligands (EGF, Transforming Growth Factor α, amphiregulin, betacellulin, heparin-binding EGF and Heregulin), basic Fibroblastic growth factors (bFGF) and other members of its super-family (FGF1, FGF4), members of Platelet-Derived Growth Factor family (PDGF AA, AB, BB), Interleukins, and members of the Transforming Growth Factor β superfamily.

The term “antiproliferative agent” is used throughout the specification to describe an agent which will prevent the proliferation of cells during methods according to the present invention which enrich pluripotent stem and/or progenitor cells. Exemplary antiproliferative agents include, for example, Ara-C, methotrexate and other proliferative agents. Preferred antiproliferative agents are those agents which limit or prevent the growth of proliferating cells within an umbilical cord blood sample or mononuclear cell fraction thereof so that quiescent stem and/or progenitor cells may be enriched.

The term “differentiation agent” or “neural differentiation agent” is used throughout the specification to describe agents which may be added to cell culture (which term includes any cell culture medium which may be used to grow differentiated cells including neural and/or other cells according to the present invention) containing pluripotent stem and/or progenitor cells which will induce the cells to a desired cellular phenotype. Preferred differentiation agents for use in the present invention include, for example, antioxidants, including retinoic acid, fetal or mature neuronal cells including mesencephalic or striatal cells or a growth factor or cytokine such as brain derived neurotrophic factor (BDNF), glial derived neurotrophic factor (GDNF) and nerve growth factor (NGF) or mixtures, thereof. Additional differentiation agents include, for example, growth factors such as fibroblast growth factor (FGF), transforming growth factors (TGF), ciliary neurotrophic factor (CNTF), bone-morphogenetic proteins (BMP), leukemia inhbitory factor (LIF), glial growth factor (GGF), tumor necrosis factors (TNF), interferon, insulin-like growth factors (IGF), colony stimulating factors (CSF), KIT receptor stem cell factor (KIT-SCF), interferon, triiodothyronine, thyroxine, erythropoietin, thrombopoietin, silencers, (including glial-cell missing, neuron restrictive silencer factor), SHC(SRC-homology-2-domain-containing transforming protein), neuroproteins, proteoglycans, glycoproteins, neural adhesion molecules, and other cell-signalling molecules and mixtures, thereof. Differentiation agents which can be used in the present invention are detailed in “Marrow-mindedness: a perspective on neuropoiesis”, by Bjorn Scheffler, et al., TINS, 22, pp. 348-356 (1999), which is incorporated by reference herein.

The term “neurodegenerative disease” is used throughout the specification to describe a disease which is caused by damage to the central nervous system and which damage can be reduced and/or alleviated through transplantation of neural cells according to the present invention to damaged areas of the brain and/or spinal cord of the patient. Exemplary neurodegenerative diseases which may be treated using the neural cells and methods according to the present invention include for example, Parkinson's disease, Huntington's disease, amyotrophic lateral sclerosis (Lou Gehrig's disease), Alzheimer's disease, lysosomal storage disease (“white matter disease” or gliaudemyelination disease, as described, for example by Folkerth, J. Neuropath. Exp. Neuro., 58, 9, Sep. 1999), Tay Sachs disease (beta hexosamimidase deficiency), other genetic diseases, multiple sclerosis, brain injury or trauma caused by ischemia, accidents, environmental insult, etc., spinal cord damage, ataxia and alcoholism. In addition, the present invention may be used to reduce and/or eliminate the effects on the central nervous system of a stroke or a heart attack in a patient, which is otherwise caused by lack of blood flow or ischemia to a site in the brain of said patient or which has occurred from physical injury to the brain and/or spinal cord. The term neurodegenerative diseases also includes neurodevelopmental disorders including for example, autism and related neurological diseases such as schizophrenia, among numerous others.

Selection of adult stem cells from any adult source, or placental or umbilical cord tissue, umbilical cord or placental blood, amniotic fluid or amnion pluripotential stem and/or progenitor cells according to the present invention can be done in a number of ways. For example, the cells may be selected using, for example a magnetic cell separator (FACS) or other system which removes all cells which contain a CD marker and then the remaining cells may be expanded in growth medium or differentiated in growth medium which includes a differentiation agent. Alternatively, an enriched population of stem and/or progenitor cells may be obtained from a sample of mononuclear cells by subjecting the cells to an agent such as Ara-C or other anti-proliferative agent such as methotrexate, which causes the death of proliferating cells within a sample (the stem and/or progenitor cells are non-proliferating and are unaffected by the agent). The remaining cells may then be grown in a cell culture medium which contains a mitogen to produce a population of differentiated and quiescent cells, which cell population may be further grown to concentrate the quiescent cells to the effective exclusion of the differentiated cells (the quiescent cells in the final cell medium will greatly outnumber the original differentiated cells which do not grow in the medium). The quiescent cells may then be induced to adopt a number of different neural and/or other phenotypes, which cells may be used directly in transplantation.

Additional in vitro differentiation techniques can be adapted through the use of various cell growth factors and co-culturing techniques known in the art. Besides co-culturing with fetal mesencephalic or striatal cells, a variety of other cells can be used, including but not limited to accessory cells, and cells from other portions of the fetal and mature central nervous system.

The term “gene therapy” is used throughout the specification to describe the transfer and stable insertion of new genetic information into cells for the therapeutic treatment of diseases or disorders. The foreign gene is transferred into a cell that proliferates to spread the new gene throughout the cell population. Thus, stem cells, or pluripotent progenitor cells according to the present invention either prior to differentiation or preferably, after differentiation to a neural or other cell phenotype, are the target of gene transfer, since they are proliferative cells that produce various progeny lineages which will potentially express the foreign gene.

The present invention therefore relates to the discovery that non-controversial stem cells which are obtained from adults (adult stem cells) or from placental tissue or umbilical cords or umbilical cord/placental blood amniotic fluid or amnion, are capable of being reprogrammed and used as substitute stem cells for controversial embryonic stem cells. This is an unexpected result.

In this first aspect of the present invention, human adult stem cells or alternatively, stem cells obtained from otherwise discarded placentas or umbilical cords themselves or alternatively, umbilical cord or placental blood or amniotic fluid or amnion, which is obtained after and as a consequence of the delivery of a baby, may be reprogrammed to produce cells which may be used in autologous procedures. The resulting stem cells, which have characteristics consistent with autologous embryonic stem cells, may develop and differentiate into any type of cell useful for treatment.

In preferred aspects of the present invention, the reprogrammed stem cells which were obtained from the placenta or umbilical cord itself or the blood of the umbilical cord or placenta or amniotic fluid or anuion are referred to as “neonic” stem cells. These stem cells, due to the reprogramming, are autologous to the source cells, as a consequence of reprogramming that simply creates embryonic stem cells from those original stem cells through exposure to a cell medium which is consistent with the makeup of the cytosol from embryonic stem cells, or alternatively, by fusing genetic material from an adult stem cell (or other adult cell) with a denucleated stem cell obtained room the placenta or umbilical cord tissue, or umbilical or placental blood or amniotic fluid or amnion to produce a neonic stem cell. The resulting reprogrammed stem cells are autologous to the host from which the reprogramming nuclear material came from and have similar characteristics with embryonic stem cells. An exciting feature of these neonic stem cells is the fact that these stem cells have characteristics consistent with embryonic stem cells, can be developed or differentiated into virtually any other cell used in treatment and this result can be obtained without the moral, legal or ethical dilemmas posed by the use of traditional embryonic stem cells.

The present invention also relates to methods of reprogramming stem cells (from adult stem cells or stem cells obtained from placenta or umbilical cords or umbilical cord or placental blood, amniotic fluid or amnion) to produce early stage stem cells which are similar in characteristics to embryonic stem cells. In a first method, human adult stem cells which may be obtained from any adult source or stem cells which are obtained directly from placenta or umbilical cords themselves or from umbilical cord or placenta blood, amniotic fluid or amnion are exposed to an effective concentration of cellular medium (embryonic stem cell medium) which contains growth factors and related nutrients of the cytosol of embryonic stem cells. In this method, the isolated stem cells are exposed to embryonic stem cell medium in an amount and for a period (preferably, at least through one generation of development, more preferably at least two or three generations or more of development) and then are isolated as reprogrammed stem cells. In the case of adult stem cells, these cells may obtain characteristics which are unexpectedly close to the phenotypic characteristics of embryonic stem cells. In the case of neonic stem cells (i.e. those stem cells which are obtained from placenta or umbilical cords or umbilical cord or placental blood or amniotic fluid or amnion and reprogrammed), these are phenotypically very close to embryonic stem cells.

In a second method aspect of the present invention, pluripotent stem cells obtained from any adult source, or placenta or umbilical cords directly, or from umbilical cord or placental blood or amniotic fluid or amnion are first denucleated (using a centrifugation step to remove the DNA material from the cells) and then the denucleated cells are fused with cells or genetic material taken from an individual to be treated, for example an adult, preferably, but not necessarily stem cells of that individual. These fused or hybrid cells obtain characteristics which are consistent with the phenotype of the pluripotent stem cells and act as pluripotent stem cells, but are autologous to the patient (have the same genotype as the individual from whom the nuclear material was taken and fused with the stem cell). The resulting hybrid stem cell or neonic stem cell may thereafter be used as a replacement for embryonic stem cells, but with the added benefit that the neonic stem cells also are essentially genotypically identical to the individual from whom the genetic material was taken and fused with the pluripotent stem cell. This results in a non-controversial stem cell which acts similar to an embryonic stem cell, limits or avoids unfavorable immunogenic responses because the stem cells are autologous (genetically identical to the host or patient) cells). Note that this approach may be taken with pluripotent stem cells or progenitor cells in order to create stem cells or progenitor cells which are particularly useful for therapy in an individual.

The reprogrammed stem cells which are obtained using the nuclear fusion technique may also be exposed to embryonic stem cell medium as discussed above in order to further reprogram the neonic cells to a level which is even closer phenotypically to an embryonic stem cell.

General Methods

Standard molecular biology techniques known in the art and not specifically described are generally followed as in Sambrook et al., Molecular Cloning: A Laboratory Manual, Cold Springs Harbor Laboratory, New York (1989, 1992), and in Ausubel et al., Current Protocols In Molecular Biology, John Wiley and Sons, Baltimore, Md. (1989). Polymerase chain reaction (PCR) is carried out generally as in PCR Protocols: A Guide To Methods and Applications, Academic Press, San Diego, Calif. (1990). Reactions and manipulations involving other nucleic acid techniques, unless stated otherwise, are performed as generally described in Sambrook, et al., 1989, Molecular Cloning: a Laboratory Manual, Cold Springs Harbor Laboratory Press, and methodology as set forth in U.S. Pat. Nos. 4,666,828; 4,683,202, 4,801,531; 5,192,659; and 5,272,057 and incorporated herein by reference. In-situ PCR in combination with Flow Cytometry can be used for detection of cells containing specific DNA and mRNA sequences (see, for example, Testoni et al. Blood 87:3822 (1996)).

Standard methods in immunology known in the art and not specifically described are generally followed as in Stites et al. (eds), BASIC AND CLINICAL IMMUNOLOGY, 8'th Ed., Appleton & Lange, Norwalk, Conn. (1994); and Mishell and Shigi (eds), Selected Methods In Cellular Immunology, W.H. Freeman and Co., New York (1980).

Immunoassays

In general, immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 2o 4,879,219; 5,011,771 and 5,281,521 as well as Sambrook et al., Molecular Cloning: A Laboratory Manual, Cold Springs Harbor, N.Y. (1989). Numerous other references also may be relied on for these teachings.

Antibody Production

Antibodies may be monoclonal, polyclonal or recombinant. Conveniently, the antibodies may be prepared against the immunogen or immunogenic portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Springs Harbor, N.Y. (1988) and Borrebaeck, Antibody Engineering—A Practical Guide by W. H. Freeman and Co. (1992). Antibody fragments may also be prepared from the antibodies and include Fab and F(ab′)2 by methods known to those skilled in the art. For producing polyclonal antibodies a host, such as a rabbit or goat, is immunized with the immunogen or immunogenic fragment, generally with an adjuvant and, if necessary, coupled to a carrier; antibodies to the immunogen are collected from the serum. Further, the polyclonal antibody can be absorbed such that it is monospecific. That is, the serum can be exposed to related immunogens so that cross-reactive antibodies are removed from the serum rendering it monospecific.

For producing monoclonal antibodies, an appropriate donor is hyperimmunized with the immunogen, generally a mouse, and splenic antibody-producing cells are isolated. These cells are fused to immortal cells, such as myeloma cells, to provide a fused cell hybrid that is immortal and secretes the required antibody. The cells are then cultured, and the monoclonal antibodies harvested from the culture media. Antibodies are used in the present invention to help to isolate stem cells from a sample of mononuclear cells or other cellular material.

For producing recombinant antibodies, messenger RNA from antibody-producing B-lymphocytes of animals or hybridoma is reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982). The binding of antibodies to a solid support substrate is also well known in the art. (see for a general discussion Harlow & Lane, Antibodies: A Laboratory Manual, Cold Spring Harbor Laboratory Publications, New York, 1988 and Borrebaeck, Antibody Engineering—A Practical Guide, W.H. Freeman and Co., 1992). The detectable moieties contemplated with the present invention can include, but are not limited to, fluorescent, metallic, enzymatic and radioactive markers. Examples include biotin, gold, ferritin. alkaline phosphates, galactosidase, peroxidase, urease, fluorescein, rhodamine, tritium, ¹⁴C, iodination and green fluorescent protein.

Gene Therapy

Gene therapy as used herein refers to the transfer of genetic material (e.g., DNA or RNA) of interest into a host to treat or prevent a genetic or acquired disease or condition. The genetic material of interest encodes a product (e.g., a protein. polypeptide. and peptide, functional RNA, antisense) whose in vivo production is desired. For example, the genetic material of interest encodes a hormone, receptor, enzyme, polypeptide or peptide of therapeutic value. Alternatively, the genetic material of interest encodes a suicide gene. For a review see “Gene Therapy” in Advances In Pharmacology, Academic Press, San Diego, Calif., 1997.

Administration of Cells for Transplantation

The reprogrammed stem cells of the present invention with or without further differentiation are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

In the method of the present invention, the reprogrammed stem cells or differentiated stem cells of the present invention can be administered in various ways as would be appropriate to implant into the patient, for example, in the central nervous system, cardiovascular system, blood or bone marrow, including but not limited to parenteral, including intravenous and intraarterial administration, intrathecal administration, intraventricular administration, intraparenchymal, intracranial, intracisternal, intrastriatal, and intranigral administration. In addition, all of these routes of administration may be used to effect transplantation of stem cells and cells differentiated from the stem cells of the present invention.

Pharmaceutical compositions comprising effective amounts of reprogrammed stem cells are also contemplated by the present invention. These compositions comprise an effective number of reprogrammed stem cells, optionally in combination with a pharmaceutically acceptable carrier, additive or excipient. In certain aspects of the present invention, cells are administered to the patient in need of a transplant in sterile saline. In other aspects of the present invention, the cells are administered in Hanks Balanced Salt Solution (HBSS) or Isolyte S, pH 7.4. Other approaches may also be used, including the use of serum free cellular media. Such compositions, therefore, comprise effective amounts or numbers of reprogrammed stem cells in sterile saline. These may be obtained directly by using fresh or cryopreserved umbilical cord blood or alternatively, by separating out the mononuclear cells (MNC) from the whole blood, using density gradient separation methods, among others, which are well known in the art (one such approach is presented herein). Intravenous or intraarterial administration of the stems cells in sterile saline to the patient may be preferred in certain indications, whereas direct administration at the site of or in proximity to the diseased and/or damaged tissue may be preferred in other indications. Further differentiation of the reprogrammed stem cells may be advised, depending upon the treatment to be provided.

In using compositions according to the present invention, fresh or cryopreserved adult stem cells from any adult source, or placenta or umbilical cord tissue, placental or umbilical cord blood, or amniotic fluid or amnion tissue, a mononuclear fraction thereof, or fractions wherein stems cells are isolated and/or concentrated (using FACS or other separation methods for isolating pluripotent stem or progenitor cells from a population of mononuclear cells) may be used.

The reprogrammed stem cells may be used without treatment with a differentiation agent or with an effective amount of a differentiation agent prior to being used for treatment, for example, in a neuronal transplant or other tissue transplant.

In aspects of the invention in which adult stem cells from any adult source, or placenta or umbilical cord tissue stem cells or placental or cord blood stem cells, or amniotic fluid or amnion stem cells are differentiated, the use of standard media which has been supplemented with at least one or more differentiation agent is preferred. A combination of retinoic acid and nerve growth factor (NGF) and/or other growth factor in effective amounts in certain aspects of the present invention as the differentiation agent is preferred. In certain preferred aspects of the present invention, neural and/or other cells are prepared from adult stem cells from any adult source, or cord blood stems cells growing in standard growth media in a two step approach using neural and/or other proliferation media followed by a differentiation media. In this aspect of the present invention, cells grown in standard cellular media (preferably, at least a minimum essential medium supplemented with non-essential amino acids, glutamine, mercaptoethanol and fetal bovine serum (FBS)) are initially grown in a “neural and/or other proliferation medium” (i.e., a medium which efficiently grows neural and/or other cells) followed by growth in a “differentiation medium”) (generally, similar to the neural proliferation medium with the exception that specific nerve differentiation agents are added to the medium and in certain cases, other growth factors are limited or removed). A preferred proliferation medium (neural and/or other cells) is a media which contains DMEM/F12 1:1 cell media, supplemented with 0.6% glucose, insulin (25 μml), transferrin (100 μg/ml), progesterone 20 nM, putrescine (60 μM, selenium chloride 30 nM, glutamine 2 mM, sodium bicarbonate 3 mM, HEPES 5 mM, heparin 2 μg/ml and EGF 20 nm/ml, bFGF 20 ng/ml. One of ordinary skill will readily recognize that any number of cellular media may be used to provide appropriate proliferation medium and/or differentiation medium (neural and/or other cells).

The following examples are provided to illustrate or exemplify certain preferred embodiments of the present invention illustrative of the present invention but are not intended in any way to limit the present invention.

Preparation of Cellular Samples

Cryopreserved or fresh placental or umbilical cord tissue, or placental or umbilical cord blood (from human umbilical cord that remains attached to placenta after delivery), or amniotic fluid or amnion is harvested and processed by Ficoll centrifugation. This results in nearly 100% recovery of mononuclear cells which can be a) processed into sub-populations based on surface markers or b) cryopreserved for later use. Alternatively, stem cells may be obtained directly from umbilical cords (i.e. the actual cord and/or placental or amnion tissue, not the blood) or from adult blood or other tissue to provide usable stem cells which may be reprogrammed according to the present invention.

While the invention has been described hereinabove, care should be taken not to limit the invention in a manner which is unintended and is inconsistent with the invention as set forth in the following claims.

1. Reprogrammed stem cells obtained by exposing adult pluripotent stem or progenitor cells or pluripotent stem or progenitor cells obtained from placental or umbilical cord tissue or placental or umbilical cord blood or amniotic fluid or amnion exposed to
a. an effective amount of embryonic stem cell medium or
b. a denucleation procedure to remove nuclear DNA from said stem cell followed by introduction of nuclear material from a cell of a patient to be treated.

2. The reprogrammed stem cells of claim 1 wherein said introduction occurs by fusing a patient cell with a denucleated stem cell.

3. The reprogrammed stem cells of claim 1 obtained by exposing stem cells from umbilical cord tissue to a denucleation procedure followed by introducing nuclear material from a patient cell.

4. The reprogrammed stem cells of claim 1 obtained by exposing stem cells from umbilical cord or placental blood to a denucleation procedure followed by introducing nuclear material from an adult cell into said denucleated stem cell.

5. The reprogrammed stems cells of claim 1 obtained by exposing stem cells from amniotic fluid or amnion to a denucleation procedure followed by introducing nuclear material from an adult cell into said denucleated cell.

6. The reprogrammed stem cells of claim 1 wherein said embryonic stem cell medium is a minimum essential medium further comprising at least one growth factor, glucose, non-essential amino acids, insulin and transferrin.

7. The reprogrammed cells of claim 6 wherein said growth factor is fibroblast growth factor, transforming growth factor beta or mixtures thereof and said medium further comprises at least one additional component selected from the group consisting gamma amino butyric acid, pipecholic acid, lithium and mixtures thereof.

8. A method of reprogramming stem cells to produce non-controversial stem cells said method comprising:
a. providing a sample comprising at least one stem cell obtained from an adult, from placental umbilical cord tissue or from umbilical cord or placental blood or from amniotic fluid or amnion tissue; and
b. exposing said sample to an effective amount of embryonic stem cell medium.

9. The method according to claim 8 wherein said sample in step a is obtained from placenta or umbilical cord tissue.

10. The method according to claim 8 wherein said sample in step a is obtained from umbilical cord or placental blood.

11. The method according to claim 8 wherein said sample in step a is obtained from amniotic fluid or amnion tissue.

12. The method according to claim 8 wherein said embryonic stem cell medium is a minimum essential medium further comprising at least one growth factor, glucose, non-essential amino acids, insulin and transferrin.

13. The method according to claim 12 wherein said growth factor is fibroblast growth factor, transforming growth factor beta or mixtures thereof and said medium further comprises at least one additional component selected from the group consisting gamma amino butyric acid, pipecholic acid, lithium and mixtures thereof.

14. The method according to claim 13 wherein said growth factor is a mixture of fibroblast growth factor and transforming growth factor beta.

15. A method of reprogramming stem cells to produce non-controversial stem cells said method comprising:
a. providing a sample comprising at least one stem cell obtained from an adult, from placental or umbilical cord tissue or from umbilical cord or placental blood, or from amniotic fluid or amnion tissue;
b. denucleating said stem cell from step a; and
c. introducing DNA from a patient into said denucleated stem cell.

16. The method according to claim 15 wherein said sample in step a is obtained from placental or umbilical cord tissue.

17. The method according to claim 8 wherein said sample in step a is obtained from umbilical cord or placental blood.

18. The method according to claim 8 wherein said sample in step a is obtained from amniotic fluid or amnion tissue.

19. The method according to claim 15 wherein said denucleating step occurs by centrifugation.

20. The method according to claim 15 wherein said DNA is introduced into said denucleated stem cell by fusion with a cell from a patient to be treated.

21. A method of treating a patient for a disease state or condition treatable with stem cells, said method comprising administering to said patient effective amounts of reprogrammed stem cells according to claim 1.

22. The method according to claim 21 wherein said disease state or condition is cancer.

23. The method according to claim 22 wherein said cancer is leukemia.

24. The method according to claim 21 wherein said disease state or condition is a neurodegenerative disorder, a brain or spinal cord injury or a neurological deficit.

25. The method according to claim 21 wherein said disease state or condition is Parkinson's disease, Huntington's disease, multiple sclerosis (MS), Alzheimer's disease, Tay Sach's disease, lysosomal storage disease, or a brain or spinal cord injury.

26. A method of effecting hematopoietic reconstitution in a patient in need thereof, comprising administering to said patient an effective amount of reprogrammed stem cells according to claim 1.

27. The method according to claim 26 wherein the patient has an HIV infection or AIDS.
